(19) United States
(12) Patent Application Publication
(10) Pub. No.: US 2022/0162687 A1
(43) Pub. Date: May 26, 2022

(54) NOVEL SYSTEMS, METHODS AND COMPOSITIONS FOR THE DIRECT SYNTHESIS OF STICKY ENDED POLYNUCLEOTIDES

(71) Applicant: THE REGENTS OF THE UNIVERSITY OF COLORADO, A BODY CORPORATE, Denver, CO (US)

(72) Inventors: Brian DEDECKER, Boulder, CO (US); Madison L. ADAMTHWAITE, Broomfield, CO (US); Nathaniel Z. MOORE, Boulder, CO (US); Lauren A. VANHAUSEN, Broomfield, CO (US); Mckayla T. VLASITY, Boulder, CO (US)

(21) Appl. No.: 17/616,958

(22) PCT Filed: Jun. 8, 2020
(86) PCT No.: PCT/US2020/036645

§ 371 Date: Dec. 6, 2021

Related U.S. Application Data
(60) Provisional application No. 62/858,163, filed on Jun. 6, 2019.

(51) Int. Cl. C12Q 1/6853; C12P 19/34; C12N 9/00

(52) U.S. Cl. CPC C12Q 1/6853; C12Y 605/01001; C12N 9/93; C12P 19/34

(57) ABSTRACT

The current inventive technology includes systems, methods, and compositions for directly synthesizing sticky ended DNA fragments for subsequent gene assembly. In a preferred embodiment, the inventive technology includes strategies for the direct synthesis of sticky ended DNA with 5′ overhangs that have any desired length and base composition, using typical PCR protocols with no additional manipulation. In another embodiment, the inventive technology includes the direct synthesis of sticky ended DNA using chemically modified oligonucleotide primers in a polymerase chain reaction (PCR). In certain embodiments, the inventive technology allows for the generation of larger DNA constructs formed by the sticky-ended assemblies generally described herein compared to traditional synthesis and ligation applications.

This application claims the benefit of and priority to U.S. Provisional Application No. 62/858,163, filed Jun. 6, 2019. The entire specification and figures of the above-referenced application are hereby incorporated, in their entirety by reference.

### TECHNICAL FIELD

The inventive technology relates to the field of recombinant DNA assembly, in particular, the direct synthesis of sticky ended DNA using chemically modified primers in a polymerase chain reaction (PCR).

### BACKGROUND

Recombinant polynucleotide assembly techniques have been utilized by scientists for many decades to engineer combinations of gene sequences. For example, the use of synthetic DNA sequences goes beyond the laboratory setting and has been used to generate vaccines, human insulin, insect-repellent crops, and has progressed in the ability to manipulate bacteria to produce biofuels and synthetic plastics. More specifically, recombinant DNA assembly techniques allow for the creation of novel DNA combinations that can be used for many purposes. Gene assembly was introduced into the field with the discovery of restriction enzymes, which provided a convenient way to cleave DNA that can then be ligated into larger DNA constructs. Specifically, restriction endonucleases recognize a specific DNA sequence and cleave that DNA into a blunt or sticky ended fragment within or close to the recognition sequence. Blunt ended fragments are cleaved at the same base pair resulting in a straight cut (FIG. 1A). Alternatively, the DNA is cleaved to create an overhanging region known as a “sticky end”, where one single strand of DNA protrudes of typically 4 base pairs (FIG. 1B). Sticky ends are important as they specifically match DNA fragments to be assembled through base pairing. Restriction endonuclease can be used to generate sticky ended regions on different DNA sequences, allowing for complementary pairing of these fragments and the creation of a new DNA construct.

Restriction endonucleases allowed for the initial construction of new DNA species; however, this method has drawbacks. Restriction site sequences are introduced to specify the splice junctions, and these sequences need to be unique throughout the entire DNA sequence. Thus, it becomes impossible to find unique restriction site sequences when ligating larger gene assemblies. After ligation of the fragments, the recognition site is still present causing undesired scars in the final products, which could cause modifications to the amino acid sequences during translation. Since these initial discoveries, scientists have been searching for an efficient way to assemble DNA constructs that avoid these inherent limitations of restriction enzymes. Multiple techniques have been developed to assemble various DNA sequences, yet most prove to be inefficient and unable to assemble multiple fragments in one reaction.

For example, a fairly recent DNA construct assembly tool, Golden Gate Assembly, utilizes the characteristics of Type IIS restriction endonucleases that are able to cleave DNA in a sticky ended fashion downstream of their recognition sequences. This allows sticky ended fragments with 4 base pair overhangs to be made at any nucleotide sequence (FIG. 2). While this method rids the problem of having scars present in the final product, there are still significant limitations. Golden Gate Assembly can only create 4 base pair overhangs that are prone to mismatching. The method also requires the necessary enzyme recognition site to be present at precise locations where the overhangs are desired. The restriction site must also be unique to the plasmid and not in the insert sequence. If the restriction site is not present, it could be introduced via PCR, as generally outlined in FIG. 4, with primers that contain the desired enzymatic site. However, this no longer gives scientists an easy, fast procedure and limits the number of fragments that can be assembled in one reaction.

Another technique, named Gibson Assembly, is widely used by biologists. In this method, overlapping regions of double stranded DNA fragments can be used to assemble DNA products in a one-tube, one-step reaction. Exonuclease is used to chew back the 5′ regions of each DNA fragment, exposing a complementary single stranded region of DNA. The strands are then ligated together and the gaps are filled in with polymerase, producing a double stranded DNA fragment (FIG. 3). While Gibson Assembly provides a one-step solution to recombinant DNA technology, it also requires skillful design of large, 20-80 base pair overlaps. There are also significant difficulties with the use of the exonuclease and polymerase in a one-step reaction resulting in decreased enzymatic efficiency due to the conflicting kinetics between molecules. Gibson Assembly also holds limitations with the size of DNA fragments that can be used as the activity of the exonuclease is uncontrolled, so small DNA fragments cannot be used.

As can be seen, there is a need for a simple and cost-effective method that can make gene assembly easier for scientists without the limitations or difficulties of current techniques. Importantly, such an improved method would allow for increased numbers of DNA fragments and increased lengths of DNA fragments to be assembled in one reaction. As described below, the present inventors have demonstrated a novel approach to recombinant DNA assembly with the sole use of chemically modified primers, standard polymerase chain reaction (PCR) protocols, and ligation methods. The methodology is fast, simple, and effective compared to other methods allowing for an improved means to create recombinant DNA. This technique is important as synthetic biology, the creation of biological systems that do not exist in the natural world, becomes a major aspect in the future of research, human health, and environmental sustainability.

### SUMMARY OF THE INVENTION

One aspect of the current inventive technology includes systems, methods, and compositions for synthesizing recombinant DNA and in particular, directly synthesizing a sticky ended DNA oligonucleotide. In a preferred embodiment, the inventive technology includes strategies for the direct synthesis of sticky ended DNA fragments at any location with any desired length of overhang, using typical PCR protocols with no additional manipulation.

Another aspect of the current inventive technology includes systems, methods, and compositions for directly synthesizing a DNA oligonucleotide having a 5′ overhanging sticky end without the use of restriction enzymes, therefore eliminating the need for site-specific recognition sequences. Another aspect of the current inventive technology includes systems, methods, and compositions for directly synthesizing a DNA oligonucleotide having a 5′ overhanging sticky end through the use of chemically modified DNA primers. Another aspect the current inventive technology includes systems, methods, and compositions for a novel method of directly synthesizing a DNA oligonucleotide having a 5′ overhanging sticky ended DNA fragment using a modified polymerase chain reaction (mPCR) protocol with chemically modified primers. Another aspect of the current inventive technology includes systems, methods, and compositions directly synthesizing as DNA oligonucleotide having a 5′ overhanging sticky ended DNA wherein the single stranded overhang product is customizable in its sequence, location, and length.

Another aspect the current inventive technology includes systems, methods, and compositions for a novel method of directly synthesizing a DNA oligonucleotide having a 5′ overhanging sticky ended DNA using a standard polymerase chain reaction (PCR) protocol with chemically modified oligonucleotide primers. Another aspect of the invention includes chemically modified oligonucleotide primers, or blocking primers, that prevent the elongation and/or the exonuclease activity of a polymerase. In one embodiment, the chemically modified oligonucleotide primers, or blocking primers, may sterically hinder a polymerase preventing its elongation and/or the exonuclease activity.

Another aspect of the invention includes systems, methods, and compositions for the use of chemically modified forward and reverse primers in a PCR to create amplified, sticky ended DNA fragments that can further be used in transformation procedures to create functional DNA constructs. Another aspect of the invention include systems, methods, and compositions for the use of chemically modified forward and reverse primers in a PCR to create amplified, sticky ended DNA fragments that can further be used in ligation procedures to create functional DNA constructs. In one preferred embodiment, a chemical modification, or blocking group, may be coupled to a nucleoside, or the DNA phosphate backbone, on the primer. Another aspect of the invention include systems, methods, and compositions for the use of chemically modified forward and reverse primers and unmodified primers in a PCR to create amplified, sticky ended DNA fragments that can further be used in transformation procedures to create functional DNA constructs. Another aspect of the invention include systems, methods, and compositions for the use of chemically modified forward and reverse primers and unmodified primers in a PCR to create amplified, sticky ended DNA fragments that may be chemically modified through the application of a kinase and further litigated with a ligation enzyme.

Another aspect of the invention include systems, methods, and compositions for the use of chemically modified forward and reverse primers in a PCR to create amplified, sticky ended DNA fragments wherein the chemical modification, or blocking modification on the phosphate group, includes the thermally labile group 4-oxotetradec-1-yl (OXP) phosphate group modification on any desired deoxynucleotide of the primer.

Another aspect of the invention includes systems, methods, and compositions for the use of chemically modified forward and reverse primers in a PCR to create amplified, sticky ended DNA fragments wherein the chemical modification, or blocking modification includes a photocage as a blocking group attached to the DNA phosphate backbone, sugar or base. Another aspect of the invention include systems, methods, and compositions for the use of chemically modified forward and reverse primers in a PCR to create amplified, sticky ended DNA fragments wherein the chemical modification, or blocking modification, includes a 1-(4,5-dimethoxy-nitrophenyl) diazoethane (DMNPE) as a caging group attached to the DNA phosphate backbone.

Another aspect of the invention includes systems, methods, and compositions for the use of chemically modified forward and reverse primers, and unmodified primers, in a PCR to create amplified, sticky ended DNA fragments that may be ligated by a host in vivo by its endogenous DNA repair machinery.

In another preferred aspect, the invention includes systems, methods, and compositions for the use of chemically modified forward and reverse primers, and unmodified primers, in a PCR or other analogous system, to create amplified, sticky ended DNA fragments that may be ligated in vitro, or by a host in vivo by its endogenous DNA repair machinery, wherein the DNA fragments may form constructs that are larger than traditional DNA constructs produced in similar systems without the inventive technology.

Another aspect of the invention includes systems, methods, and compositions for the use of chemically modified forward and reverse primers, and unmodified primers, in a PCR to create amplified, sticky ended DNA fragments that can further be used in ligation procedures to create functional DNA constructs. In one preferred embodiment, a chemical modification or blocking group may be coupled to a nucleoside, or the DNA phosphate backbone, on the primer. In additional aspects, a chemical modification or blocking group may be removed, or deprotected, through one or more processes including, but not limited to: enzymatic deprotection, thermal deprotection, chemical deprotection, catalytic deprotection, photocage deprotection, or other reversible chemistry.

Another aspect of the invention may include the use of various reversible chemistries that may be used to modify an oligonucleotide such that it blocks the conventional polymerase-DNA mechanism in one embodiment thereof. The reversible chemistries compositions may act as a reversible blocking group modification that prevents DNA polymerase from fully extending the complementary strand during PCR amplification resulting in a 5′ overhang. In one preferred aspect, variations of the “trimethyl lock” composition having various R trigger groups may be coupled with an oligonucleotide or oligonucleotide subunit, such as a phosphoramidite, and may act as specific triggers to remove the compound from the oligonucleotide. Specific R groups can be triggered for removal of the entire group by chemicals, photons or enzymatically.

Another aspect of the invention may include the generation of coupling of a halide (I, Br & Cl) to a reversible chemistry composition, such as a trimethyl lock forming an alkyl halide. The alkyl halide can then be attached to the previously synthesized oligonucleotide that contains a thiophosphate group at specific locations, and may be coupled with an oligonucleotide subunit, such as a phosphoramidite. The alkyl halide may act as a reversible blocking group modification that prevents DNA polymerase from fully extending the complementary strand during PCR amplification resulting in a 5′ overhang.

Further objects of the inventive technology will become apparent from the description and drawings below.

### BRIEF DESCRIPTION OF DRAWINGS

FIG. 1 A&B: Examples of restriction endonucleases. Cut sites are indicated by the blue arrows. (A) Example of a restriction endonuclease that cleaves the DNA in a blunt ended fashion. The result is two DNA fragments with no overhanging regions. (B) Example of a restriction endonuclease that cleaves DNA in a sticky ended fashion. The result is overhanging regions indicated in red.

FIG. 2: Example of Golden Gate Assembly. A type IIS restriction enzyme has a specific recognition sequence. The enzyme then cleaves downstream of the recognition sequence, indicated by the blue arrows. The result of the digest is a sticky ended DNA fragment. The fragment can then ligate to other complementary DNA fragments resulting in a recombinant DNA product.

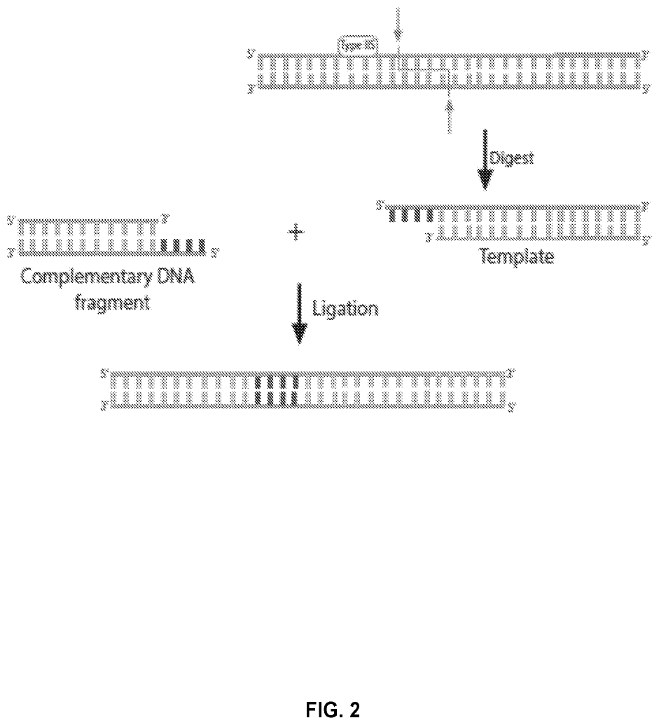

FIG. 3: Example of Gibson Assembly. Two double stranded DNA fragments are designed to have optimal overlapping regions, indicated in blue. An exonuclease is used to degrade the 5′ ends of the DNA leaving a complementary overhanging region. The fragments are ligated together at the complementary base pairs and the gaps are filled in by polymerase. The result is recombinant double stranded DNA.

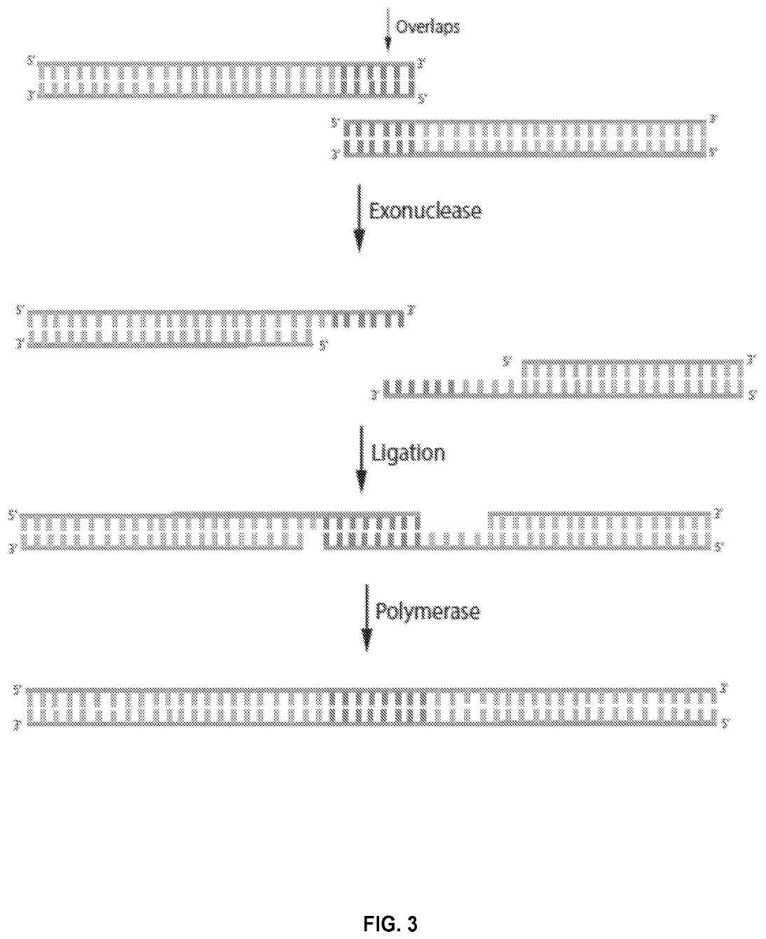

FIG. 4: Polymerase chain reaction overview and example. The double stranded DNA template, that is desired to be amplified, is placed in high heat conditions causing the complementary DNA strands to denature. The reaction is then cooled, allowing two primers to anneal to the 3′ ends of the template DNA strands. The reaction is heated to optimal polymerase activity temperature where the polymerase binds to the free 3′-OH of the primers. The polymerase then adds dNTPS in a 5′ to 3′ fashion, making DNA that is complementary to the template strand. The product is DNA strands that are identical to the template DNA. The reaction will then be heated again, for various cycles, allowing for DNA amplification.

FIG. 5: A PCR reaction using chemically modified primers to produce 5′ sticky ended DNA fragments. Two primers are necessary for the PCR. One primer was made by standard methods and the other was made with an OXP modification, indicated by the yellow box. The polymerase extends from the free 3′-OH end of Primer 1 and becomes sterically blocked by the OXP group on Primer 2, causing the polymerase to fall off the DNA. The result of the PCR is amplified, sticky ended DNA. An insert, with a complementary overhang to the template, is ligated resulting in double stranded recombinant DNA.

FIG. 6: Exemplary blocking primer having a 4-oxo-1-tetradecanyl (OXP) structure: (a) Structure of OXP; and (b) OXP group attached to the DNA phosphate backbone.

FIG. 7: Proposed mechanism for OXP reactivity. (A) The PCR solution contains a buffer with acid-base components. The base may be interacting with the OXP modification at high temperatures. The base is a reactive species that accepts a hydrogen molecule. Removing the hydrogen from the OXP group results in the formation of a carbanion. (B) The unstable carbanion can quickly transfer electrons to form a more stable double bond; however, this results in electrons being pushed onto a highly electronegative oxygen molecule. The negative oxygen is a reactive species that can form a favorable cyclic structure causing dissociation of the OXP group from the DNA phosphate backbone.

FIG. 8: Polymerase chain reaction thermocycler conditions. Standard PCR conditions are indicated in black. The experimental PCR conditions with OXP modified primers, indicated in blue dashed lines, have lower temperatures, decreased times, and decreased cycles compared to standard PCR conditions.

FIG. 9: PCR products analyzed in the mass spectrometry data, a corresponding visual and the calculated molecular weight of the product.

FIG. 10: Mass spectrometry data shown for a PCR with 20 reaction cycles. (a) Fraction one. Analysis revealed a major product of n−1 truncation (nucleotide addition halting on the complementary strand at the first nucleotide after OXP modification). Other major products included complementary full length reverse product and forward product without OXP attached. (b) Fraction two. Analysis revealed a high intensity forward product with OXP still attached, which is a complementary product to the n−1 truncation.

FIG. 11: Graphical representation of relative amounts of PCR products detected in mass spectrometry analysis. Cycles 5, 10, 15, 20, and 25 are shown; corresponding PCR products are shown below graph.

FIG. 12: Proof of concept schematic to create sticky ended PCR products that can subsequently be ligated together. In this embodiment, a plasmid was sectioned into two parts with nucleotide gaps, one within the chloramphenicol resistance (indicated in the plasmid as a purple line). The two DNA fragments were flanked by overhanging OXP modified primers. A PCR with optimal conditions was performed resulting in DNA fragments with 6 base pair sticky ended regions. The complementary sticky ended regions were ligated together, re-forming the plasmid, and re-introducing the nucleotide gaps and plasmid resistance. The plasmid was transformed into E. coli. The sticky ended ligation was confirmed by colony growth on chloramphenicol plates.

FIG. 13: Gel electrophoresis of PCR products. Product 1 and product 2 each indicate a half of the plasmid. Controls were run using non-modified PCR primers. Gel indicated successful amplification of the template with both standard primers and OXP modified primers. PCR conditions were as follows: 95° C. for 0 seconds, 55° C. for 0 seconds, 72° C. for 5 seconds. PCRs were run at either 6 cycles (8.5 minutes) or 10 cycles (15 minutes). The gel indicated about 3.1 ng/u1 of product formed.

FIG. 14: Schematic for ligation efficiency. The PCR products had 3′ and 5′ hydroxyl groups. A kinase enzyme was used to convert 5′ hydroxyl groups to phosphate groups. Ligation, using a T7 ligase, was then able to occur resulting in a fully functional plasmid.

FIG. 15: Schematic for directly synthesizing DNA with 5′ overhang sticky ends for gene assembly.

FIG. 16 A-D: Examples of reversible chemistries that may be used to modify an oligonucleotide such that it blocks the conventional polymerase-DNA mechanism in one embodiment thereof. (A) Variations of the “trimethyl lock” with various R groups that act as specific triggers to remove the compound from the oligonucleotide. Specific R groups can be triggered for removal of the entire group by chemicals, photons or enzymatically. (B) An example of a reversible chemistry that is removed by thioesterase activity, (C) An example of a reversible chemistry that is removed by esterase activity. (D) An example of a reversible chemistry that is removed by beta-glucuronidase activity.

FIG. 17 A-B: Shows examples of methods for attaching reversible chemistries that may be used to modify an oligonucleotide such that it blocks the conventional polymerase-DNA mechanism in one embodiment thereof. (A) The reversible chemistry is attached through modified phosphoramidites incorporated during oligonucleotide synthesis. One or multiple sites may be modified on the oligonucleotide. The reversible chemistry may be attached with specific stereochemistry or as a racemic mixture. (B) The reversible chemistry is attached through nucleophilic substitution reaction directly to the synthesized oligonucleotide. A halide (I, Br & Cl) is first attached to the reversible chemistry (R). The alkyl halide can then be attached to the previously synthesized oligonucleotide that contains a thiophosphate group at specific locations. The reversible chemistry is then attached to the oligonucleotide through a nucleophilic substitution reaction. One or multiple sites may be modified on the oligonucleotide. The reversible chemistry may be attached with specific stereochemistry or as a racemic mixture.

### DETAILED DESCRIPTION OF INVENTION

The present invention includes a variety of aspects, which may be combined in different ways. The following descriptions are provided to list elements and describe some of the embodiments of the present invention. These elements are listed with initial embodiments, however, it should be understood that they may be combined in any manner, and in any number, to create additional embodiments. The variously described examples and preferred embodiments should not be construed to limit the present invention to only the explicitly described systems, techniques, and applications. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

One embodiment of the current inventive technology includes systems, methods, and compositions for synthesizing recombinant DNA, and in particular, directly synthesizing a sticky ended DNA oligonucleotide. In a preferred embodiment, the inventive technology includes strategies for the direct synthesis of sticky ended DNA, in this embodiment sticky ended DNA having 5′ overhanging DNA fragments at any location with any desired length and sequence of overhang, using typical PCR protocols with no additional manipulation.

Another embodiment of the current inventive technology includes systems, methods, and compositions for directly synthesizing DNA having a 5′ overhanging sticky end without the use of restriction enzymes, therefore eliminating the need for site specific recognition sequences. Another of the current inventive technology includes systems, methods, and compositions for directly synthesizing DNA having a 5′ overhanging sticky end through the use of chemically modified blocking oligonucleotide primers. Another embodiment the current inventive technology includes systems, methods, and compositions for a novel method of directly synthesizing DNA having a 5′ overhanging sticky ended DNA using a modified PCR protocol with chemically modified oligonucleotide primers. Another embodiment of the current inventive technology includes systems, methods, and compositions directly synthesizing DNA having a 5′ overhanging sticky ended DNA wherein the single stranded overhanging product is customizable in its sequence, location, and length.

Another embodiment the current inventive technology includes systems, methods, and compositions for a novel method of directly synthesizing DNA having a 5′ overhanging sticky ended DNA using a standard polymerase chain reaction (PCR) protocol with chemically modified oligonucleotide primers. Another embodiment of the invention includes chemically modified oligonucleotide primers, or blocking primers, that prevent the elongation and/or the exonuclease activity of a polymerase. In one embodiment, the chemically modified primers, or blocking primers, may sterically hinder a polymerase preventing its elongation and/or the exonuclease activity. Another embodiment of the invention includes systems, methods, and compositions for the use of chemically modified forward and reverse primers in a PCR to create amplified, sticky ended DNA fragments that can further be used in transformation procedures to create functional DNA constructs.

Another embodiment of the invention includes systems, methods, and compositions for the use of chemically modified forward and reverse primers in a PCR to create amplified, sticky ended DNA fragments that can further be used in ligation procedures to create functional DNA constructs. In one preferred embodiment, a chemical modification or blocking group may be coupled to a nucleoside or the DNA phosphate backbone on the primer. Another embodiment of the invention include systems, methods, and compositions for the use of chemically modified forward and reverse primers and unmodified primers in a PCR to create amplified, sticky ended DNA fragments that can further be used transformation procedures to create functional DNA constructs.

Another embodiment of the invention include systems, methods, and compositions for the use of chemically modified forward and reverse primers, and unmodified primers, in a PCR to create amplified, sticky ended DNA fragments that may be chemically modified through application of a kinase and further litigated with a ligation enzyme. Another embodiment of the invention include systems, methods, and compositions for the use of chemically modified forward and reverse primers in a PCR to create amplified, sticky ended DNA fragments wherein the chemical modification, or blocking modification includes a 4-oxotetradec-1-yl (OXP) phosphate group modification or any chemical modification on the DNA phosphate backbone, sugar or base that blocks polymerase extension

Another embodiment of the invention include systems, methods, and compositions for the use of chemically modified forward and reverse primers in a PCR to create amplified, sticky ended DNA fragments wherein the chemical modification, or blocking modification, includes a photocage as a blocking group attached to the DNA phosphate backbone. Another embodiment of the invention include systems, methods, and compositions for the use of chemically modified forward and reverse primers in a PCR to create amplified, sticky ended DNA fragments wherein the chemical modification, or blocking modification, includes a 1-(4,5-dimethoxy-nitrophenyl) diazoethane (DMNPE) as a caging group attached to the DNA phosphate backbone.

Another embodiment of the invention includes systems, methods, and compositions for the use of chemically modified forward and reverse primers and unmodified primers in a PCR to create amplified, sticky ended DNA fragments that may be ligated by a host in vivo by its endogenous DNA repair machinery. Another embodiment of the invention includes systems, methods, and compositions for the use of chemically modified forward and reverse primers, and unmodified primers, in a PCR to create amplified, sticky ended DNA fragments that can further be used in ligation procedures to create functional DNA constructs. In one preferred embodiment, a chemical modification, or blocking group, may be coupled to a nucleoside or the DNA phosphate backbone on the primer. In additional embodiments, a chemical modification or blocking group may be removed, or deprotected, through one or more processes including, but not limited to: enzymatic deprotection, thermal deprotection, chemical deprotection, catalytic deprotection, photocage deprotection, or other reversible chemistry.

In one preferred embodiment, the inventive technology includes systems, methods, and compositions for the direct synthesis of polynucleotides, preferably DNA polynucleotides that may be amplified using PCR or other like protocols and wherein such DNA products may include a sticky ended portion. In this preferred embodiment, the synthesized DNA products may include a 5′ sticky end overhanging region of a customizable length and sequence. Such 5′ sticky end overhanging regions may be complementary to one another such that they may be hybridized and/or litigated to form a recombinant DNA product.

In the embodiment, a chemically modified primer or blocking primer may be incorporated into a PCR or other similar DNA replication process. In one preferred embodiment, a blocking primer may be generated through the addition of one or more blocking groups to the primer. This blocking group may be positioned at any location along the primer and may be further coupled with the deoxynucleosides or the primers phosphate backbone.

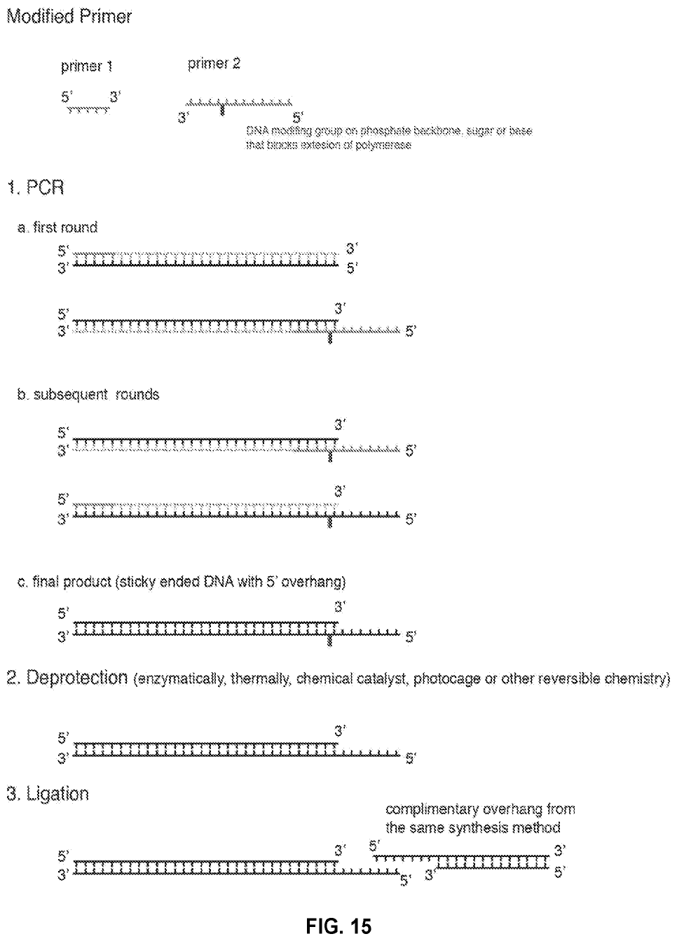

As generally shown in FIG. 15, in a preferred embodiment, a blocking primer may include a chemical blocking group, such as an OXP as a phosphate primer modification. As also shown in this embodiment, the chemical blocking group may be positioned closer to the 3′ end of the primer such that the remaining base pairs extending to the 5′ prime end, as shown below, may form the 5′ overhanging region of the sticky ended DNA product. As noted elsewhere, the length and base composition of the 5′ overhanging region of the sticky ended DNA product may be directly designed by a user depending on the specific application or need. In one embodiment, the present invention may include an OXP as a phosphate primer modification that may be used to generate products in an efficient PCR protocol with shortened times, temperatures, and cycles. The use of blocking primers, and preferably OXP modified primers, in PCR may result in a truncation relative to the phosphate group modification.

In one preferred embodiment, the present invention may include the modified primers in PCR to create exemplary 5′ overhanging products that consist of 6 base pairs, giving a 16 fold increase in annealing specificity as compared to the 4 base pair overhangs created in current methods. 5′ overhanging products can consist of any desired base pair length overhang. As noted above, in certain embodiments, the length and sequence may be customized. For example, in certain embodiments, modified primers may be used with standard or customized PCR protocols to create exemplary 5′ overhanging polynucleotide products that may be selected from the group consisting of: a 5′ overhanging polynucleotide product that consist of 1 to 4 base pairs; a 5′ overhanging polynucleotide product that consist of 1 to 6 base pairs; a 5′ overhanging polynucleotide product that consist of 1 to 10 base pairs; a 5′ overhanging polynucleotide product that consist of 1 to 20 base pairs; a 5′ overhanging polynucleotide product that consist of 1 to 30 base pairs; a 5′ overhanging polynucleotide product that consist of 1 to 40 base pairs; a 5′ overhanging polynucleotide product that consist of 1 to 50 base pairs; a 5′ overhanging polynucleotide product that consist of 1 to 60 base pairs; a 5′ overhanging polynucleotide product that consist of 1 to 70 base pairs; a 5′ overhanging polynucleotide product that consist of 1 to 80 base pairs; a 5′ overhanging polynucleotide product that consist of 1 to 90 base pairs; a 5′ overhanging polynucleotide product that consist of 1 to 100 base pairs; a 5′ overhanging polynucleotide product that consist of more than 100 base pairs.

In another preferred embodiment, the present invention may include the use of modified primers in PCR to create exemplary 5′ overhanging polynucleotide products that may be further ligated. In one preferred embodiment, a plurality of sticky ended DNA strands may be directly synthesized from a PCR, with no subsequent endonuclease digestions, and specifically ligated together, for example, to form a functional gene or expression vector and the like.

In another preferred embodiment, the present invention may include the use of modified primers in PCR to create exemplary 5′ overhanging polynucleotide products that may be used to transform one or more host organisms, such a bacterial, or other eukaryotic cell, and further ligated in vivo by the cell's endogenous DNA repair machinery. In another preferred embodiment, the present invention may include the generation of one or more chemically modified or blocking primers. Examples of the blocking modification or group that can be coupled, and or removed from a modified primer include, but are not limited to: 4-oxotetradec-1-yl (OXP) phosphate group modification or any chemical modification on the DNA phosphate backbone, sugar or base that blocks polymerase extension, for example: 1-(4,5-dimethoxy-nitrophenyl) diazoethane (DMNPE); 2-(2-nitrophenyl)propyl; a 2-(2-nitrophenyl)propyloxymethyl; a 1-(2-nitrophenyl)ethyl group; 4-oxo-1-tetradecanyl (OXP); a 6-nitropiperonyloxymethyl; an acetoxymethyl; an allyloxymethyl; a benzoyl; a benzyloxymethyl; a dimethoxybenzyloxymethyl; an isobutyryl; a methoxy; methyl phosphate; a t-butyldimethoxysilyloxymethyl; a t-butyldiphenylsilyloxymethyl; a trimethoxybenzyloxymethyl; a trityl; and an isocyanate.

Again, generally referring to FIG. 15, in one embodiment, a chemically modified primer, or blocking primer, may be paired with an unmodified primer in a PCR to directly synthesize sticky ended DNA products. In one preferred embodiment, the method may include: establishing a first unmodified primer, and a second primer having at least one chemical modification wherein the chemical modification includes the addition of a blocking group to the second primer. This blocking group may be positioned closer to the 3′ end of the blocking primer. This blocking primer may further: 1) protect the backbone phosphate group or nucleoside; and/or 2) block extension of the polymerase on a template strand. In one preferred embodiment, the chemical blocking group includes an O×P blocking modification. However, an additional preferred embodiment may include other chemical blocking groups that may protect a phosphate group on the blocking primer.

Again, referring to FIG. 15, the first primer, and the blocking primer, may be incorporated into a PCR, or other DNA amplification process. In this embodiment, at least one double stranded DNA template may undergo a first round of a PCR process resulting in the formation of two single stranded DNA (ssDNA) strands, namely a 5′ to 3′ ssDNA strand, and 3′ to 5′ ssDNA strand. The first unmodified primer may anneal or hybridize to a corresponding position on the 3′ end of a first ssDNA. The chemically modified, or blocking primer, may anneal to the corresponding 3′ end of a second ssDNA, however in this instance, a ssDNA overhanging region may extend in the 5′ direction. A polymerase will couple to the primed region and extend along the template strand forming a double stranded DNA polynucleotide (dsDNA). After this first round of PCR, the first ssDNA is now a dsDNA template with blunt ends, as would be expected in standard PCR protocols. As highlighted in FIG. 15, the second ssDNA is now a dsDNA template with a 5′ sticky-ended overhanging region.

Again, referring to FIG. 15, in second heating and annealing cycles in the PCR, such protocols being identified herein, a modified blocking primer may anneal to a 5′ ssDNA that may be extended by a polymerase forming a dsDNA template has a 5′ sticky-ended overhanging region. A first unmodified primer may anneal to a corresponding position on the 3′ end of an ssDNA template that now incorporates the blocking chemical group from the blocking primer which, in this instance, is protecting a phosphate backbone. A polymerase may attach at the primer position and begin extending the elongating strand until it reaches the blocking chemical group, which blocks polymerase extension. As a result, the polymerase disassociates from the strand forming a dsDNA product that has a 5′ sticky-ended overhanging region. As can be seen in FIG. 15, the PCR may undergo sufficient cycles to directly synthesize a desired quantity of sticky ended DNA product.

In certain embodiments, the blocking chemical group may be decoupled from the sticky ended DNA product. This deprotection step may be accomplished by cleaving the blocking protection group, in this case, from the backbone of the polynucleotide after the PCR reaction enzymatically, thermally, through a chemical catalyst, a photocage, or other reversible chemistry.

As noted above, in alternative embodiments chemical modification, or blocking modification may be made to an oligonucleotide using a variety of reversible chemistries, some of which have been described in the art. These reversible chemistries may be used to introduce chemical modification or blocking modification into a DNA oligonucleotide post-synthesis.

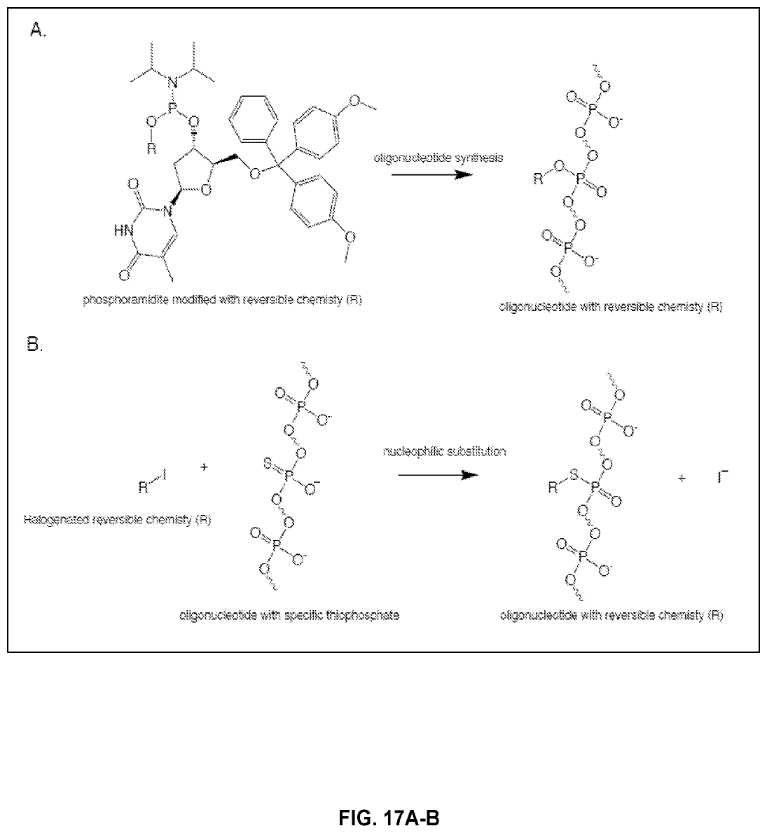

Generally referring to FIG. 17 B, by way of example and not limitation, in this embodiment, an oligonucleotide may be synthesized with an incorporated thiophosphate group at the specific position in the sequence where the reversible chemistry chemical modification, or blocking modification is to be attached. As further shown in FIG. 17B, the reversible chemistry group (R) may be formed into an alkyl halide, shown here by attaching an iodine (I) to the reversible blocking group, that may now form a reversible chemical modification, or blocking modification attached to the desired phosphate group of the DNA oligonucleotide.

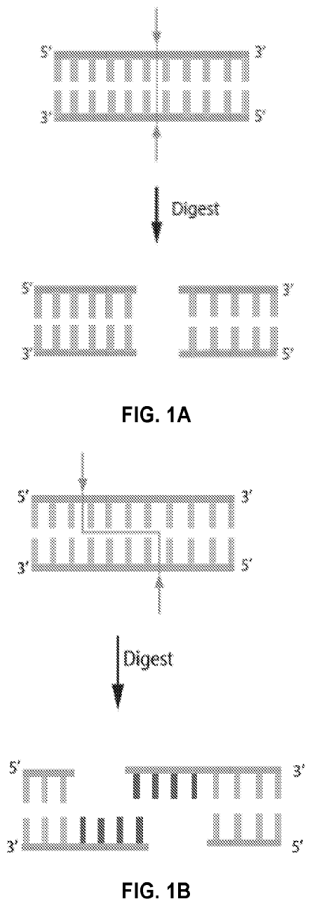

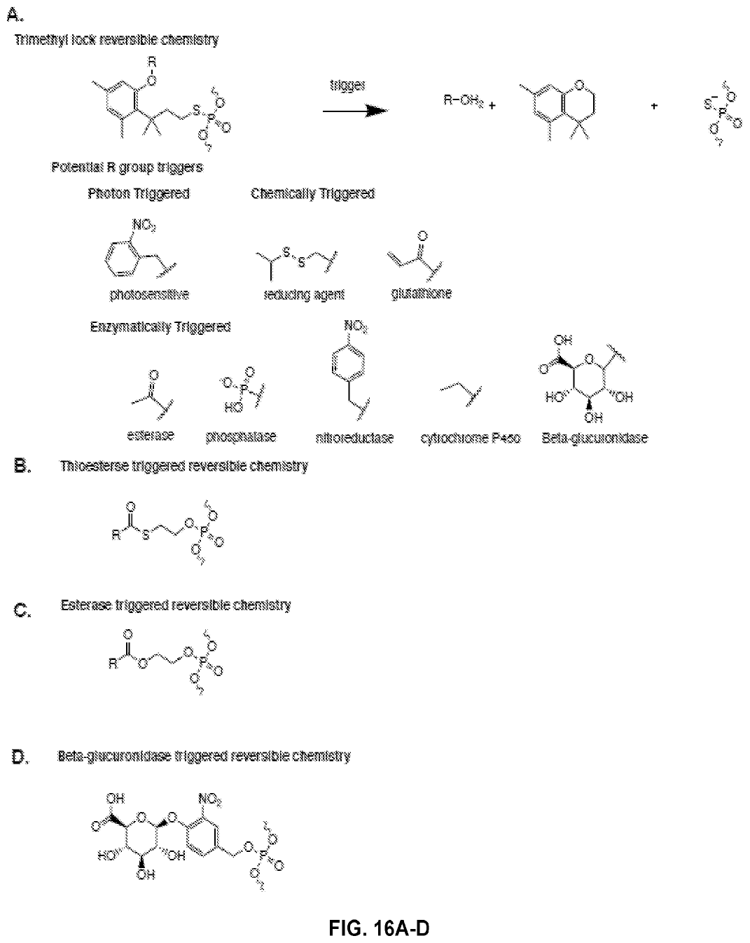

In this embodiment, a sticky ended DNA oligonucleotide may be assembled as designed, but, as described above, the ligase will not be able to bind and catalyze phosphodiester bonds between the strands until the blocking group is removed by a reversible mechanism. As shown in FIG. 16A-B, various R groups on the trimethyl lock can act as the trigger to remove the group from the oligonucleotide during the deprotection step once the PCR reaction is completed, or when desired. Specific R groups, such as those identified in FIG. 1B can be triggered for removal of the entire group. Exemplary R-group triggers may include chemically triggered R-groups, light, photon triggered R-groups, or enzymatically-triggered R-groups. As shown in FIG. 16B-D, exemplary R-group triggers may include an R-group trigger that is removed by thioesterase activity, as well as an R-group trigger may include an R-group trigger that is removed by esterase activity, as well as an R-group trigger that is removed by glucuronidase activity, and in particular beta-glucuronidase activity.

In another embodiment, the invention includes methods of coupling reversible chemistries with nucleoside phosphoramidites during oligonucleotide synthesis. As used herein, “phosphoramidites” or “nucleoside phosphoramidites” means derivatives of natural or synthetic nucleosides that are generally used to synthesize oligonucleotides, relatively short fragments of nucleic acid and their analogs.

Generally referring to FIG. 17A, an example reversible chemistry compound may be attached through modified phosphoramidites incorporated during oligonucleotide synthesis. One or multiple sites may be modified on the oligonucleotide. The reversible chemistry may be attached with specific stereochemistry or as a racemic mixture. As shown in FIG. 17B, the reversible chemistry, for example a trimethyl lock or other composition identified in FIG. 16, may be attached through nucleophilic substitution reaction directly to the synthesized oligonucleotide. As also shown in FIG. 17B, a halide may first be attached to the reversible chemistry (R). The alkyl halide can then be attached to the previously synthesized oligonucleotide that contains a thiophosphate group at specific locations. The reversible chemistry is then attached to the oligonucleotide through a nucleophilic substitution reaction. As noted in FIG. 16, in one embodiment a trimethyl lock having various R groups triggers may be used to remove the group from the oligonucleotide during the deprotection step once the PCR reaction is completed, or when desired.

In additional embodiments, the newly generated sticky ended DNA strand may be ligated with another sticky ended DNA strand that may be directly synthesized through the method generally described above. In this preferred embodiment, a sticky ended DNA strand template may be coupled with a sticky ended DNA strand insert having a complementary 5′ sticky ended overhanging region. As noted above, the length and sequence of the 5′ sticky end overhanging region may be engineered by a user. As a result, while in certain embodiments, a ligase enzyme may be used to couple the two strands, in some instances such annealing may occur in the absence of a ligating enzyme. In certain embodiments, the generated sticky ended DNA may be introduced to, for example, a host cell such as a bacteria or a eukaryotic cell where they may be ligated into specific cut sites generated by CRISPR-Cas based methods or other gene editing techniques. This novel method may be used to not only generate recombinant DNA products but efficiently transform host cells with DNA that has specifically generated sticky ends.

It is also understood that various implementations described herein can be utilized in combination with any other implementation described or disclosed, without departing from the scope of the present disclosure. Therefore, products, members, elements, devices, apparatus, systems, methods, processes, compositions, and/or kits according to certain implementations of the present disclosure can include, incorporate, or otherwise comprise properties, features, components, members, elements, steps, and/or the like described in other implementations (including systems, methods, apparatus, and/or the like) disclosed herein without departing from the scope of the present disclosure. Thus, reference to a specific feature in relation to one implementation should not be construed as being limited to applications only within said implementation.

The term “nucleic acid” or “nucleic acid molecules” include single-stranded and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), mRNA (messenger RNA), miRNA (micro-RNA), hpRNA (hairpin RNA), tRNA (transfer RNA), whether charged or discharged with a corresponding acylated amino acid), and cRNA (complementary RNA). The term “deoxyribonucleic acid” (DNA) is inclusive of cDNA, genomic DNA, and DNA-RNA hybrids. The terms “nucleic acid segment” and “nucleotide sequence segment,” or more generally “segment,” will be understood by those in the art as a functional term that includes both genomic sequences, ribosomal RNA sequences, transfer RNA sequences, messenger RNA sequences, operon sequences, and smaller engineered nucleotide sequences that encoded or may be adapted to encode, peptides, polypeptides, or proteins. In particular, nucleic acids can include, without limitation, DNA, RNA, cDNA, gDNA, ssDNA, dsDNA, or any combination thereof.

The term “primer,” or “oligonucleotide” refers to a single-stranded nucleic acid molecule of defined sequence that can base-pair to a second nucleic acid molecule that contains a complementary sequence (the “target”). The stability of the resulting hybrid depends upon the length, GC content, and the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes, primers, and oligonucleotides may be detectably-labeled, either radioactively, fluorescently, or non-radioactively, by methods well-known to those skilled in the art. dsDNA binding dyes may be used to detect dsDNA. It is understood that a “primer” is specifically configured to be extended by a polymerase, whereas a “probe” or “oligonucleotide” may or may not be so configured.

A “blocking primer” is meant to include primers that have been specifically configured to block, or hinder, the action of a polymerase. The term “blocking chemical modification” or “blocking chemical group” includes any chemical group covalently linked in a nucleic acid chain, whether a nucleoside or phosphate backbone or other binding position, and capable of blocking polymerase extension, and/or polymerase enzymatic exonuclease activity.

Also as used herein, the term “hybridization” refers to the bonding of one single-stranded nucleic acid to another single-stranded nucleic acid, such as a primer strand to a template strand, via hydrogen bonds between complementary Watson-Crick bases in the respective single-strands, to thereby generate a double-stranded nucleic acid hybrid or complex as otherwise known in the art. Commonly, the terms “hybridize,” “anneal,” and “pair” are used interchangeably in the art to describe this reaction, and so too they are used interchangeably herein. Hybridization may proceed between two single-stranded DNA molecules, two single-stranded RNA molecules, or between single-strands of DNA and RNA, to form a double-stranded nucleic acid complex.

While PCR is the amplification method used in the examples herein, it is understood that any amplification method that uses a primer may be suitable. Such suitable procedures include polymerase chain reaction (PCR); strand displacement amplification (SDA); nucleic acid sequence-based amplification (NASBA); cascade rolling circle amplification (CRCA), loop-mediated isothermal amplification of DNA (LAMP); isothermal and chimeric primer-initiated amplification of nucleic acids (ICAN); target based-helicase dependent amplification (HDA); transcription-mediated amplification (TMA), and the like. Therefore, when the term PCR is used, it should be understood to include other alternative amplification methods. For amplification methods without discrete cycles, reaction time may be used where measurements are made in cycles or Cp, and additional reaction time may be added where additional PCR cycles are added in the embodiments described herein. It is understood that protocols may need to be adjusted accordingly.

In some embodiments, PCR includes any suitable PCR method. For example, PCR can include multiplex PCR in which the polymerase chain reaction is used to amplify several different nucleic acid sequences simultaneously. Multiplex PCR can use multiple primer sets and can amplify several different nucleic acid sequences at the same time. In some cases, multiplex PCR can employ multiple primer sets within a single PCR reaction to produce amplicons of different nucleic acid sequences that are specific to different nucleic acid target sequences. Multiplex PCR can have the helpful feature of generating amplicons of different nucleic acid target sequences with a single PCR reaction instead of multiple individual PCR reactions.

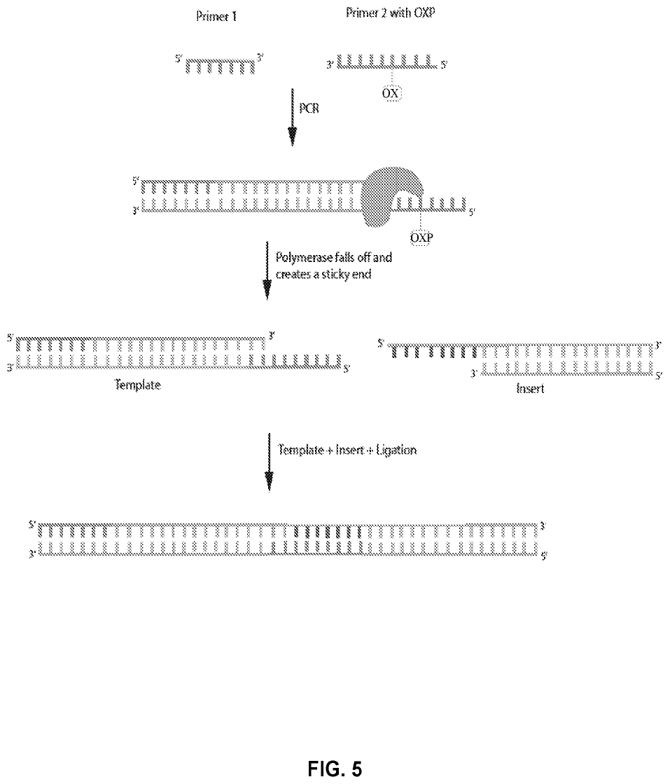

Also as used herein, the term “sticky end” or “5′ overhang” refers to double stranded DNA with any number of overhanging non-base paired bases on the 5′ end of the double stranded DNA, for example as shown generally in FIG. 5.

Also as used herein, the term ‘denaturation’ means the process of separating double-stranded nucleic acids to generate single-stranded nucleic acids. This process is also referred to as “melting”. The denaturation of double-stranded nucleic acids can be achieved by various methods, but herein it principally is carried out by heating.

Also as used herein, the term “single-stranded DNA” will often be abbreviated as “ssDNA”, the term “double-stranded DNA” will often be abbreviated as ‘dsDNA’, and the term “double-stranded RNA” will often be abbreviated as ‘dsRNA’. It is implicit herein that the term “RNA” refers to the general state of RNA which is single-stranded unless otherwise indicated.

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides. Table la, infra, contains information about which nucleic acid codons encode which amino acids.

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons).

The term “structural gene” as used herein is intended to mean a DNA sequence that is transcribed into mRNA which is then translated into a sequence of amino acids characteristic of a specific polypeptide.

The terms “approximately” and “about” refer to a quantity, level, value, or amount that varies by as much as 30%, or in another embodiment by as much as 20%, and in a third embodiment by as much as 10% to a reference quantity, level, value, or amount. As used herein, the singular form “a,” “an,” and “the” include plural references unless the context clearly dictates otherwise.

The term “trimethyl lock” A trimethyl lock is a functional group including three methyl groups in close proximity. Steric interactions between these three methyl groups promote a lactone reaction and liberation of a leaving group. A trimethyl lock may further include one or more R-group triggers that may facilitate the decoupling of the composition.

The term “reversible chemistries” or “reversible chemistry” describes broadly applicable methods for modifying molecules that include exchanging one group on a molecule for a desired functional group, such as a chemical or blocking modification on an oligonucleotide. After use, the chemical or blocking modification can be removed, or optionally can be further exchanged for a second functional group if so desired. The convenient exchange of groups is affected by reversible reactions where equilibrium conditions are controlled at each stage to direct the reaction forward or reverse as desired. After the functionalization, the addition of the functional group can be reversed by the same exchange reaction, but under different equilibrium conditions. Moreover, a functional group added to the molecule can be itself exchanged by subsequent exchange reactions.

“Thioesterase” refers to a polypeptide that can hydrolyze the thioester bond of molecules (splitting of an ester bond into acid and alcohol, in the presence of water) specifically at a thiol group.

“Esterase” refers to a hydrolase enzyme that splits esters into an acid and an alcohol in a chemical reaction with water called hydrolysis.

As used herein, “beta-glucuronidase,” “β-glucuronidase” refers to enzymes that catalyze the hydrolysis of β-D-glucuronides.

As used herein, an “alkyl” is hydrocarbon containing normal, secondary, tertiary, or cyclic carbon atoms. Unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, such as, but not limited to, ethyl, methyl, isopropyl, isobutyl, n-pentyl, n-hexyl and cyclopropylmethyl.

For example, an alkyl group can have 1 to 20 carbon atoms (i.e, C₁-C₂₀ alkyl), 1 to 8 carbon atoms (i.e., C₁-C₈ alkyl), or 1 to 6 carbon atoms (i.e., C₁-C₆ alkyl). Examples of suitable alkyl groups include, but are not limited to, methyl (Me, —CH₃), ethyl (Et, —CH₂CH₃), 1-propyl (n—Pr, n-propyl, —CH₂CH₂CH₃), 2-propyl (i—Pr, i-propyl, —CH(CH₃)₂), 1-butyl (n-Bu, n-butyl, CH₂CH₂CH₂CH₃), 2-methyl-1-propyl (i-Bu, i-butyl, —CH₂CH(CH₃)₂), 2-butyl (s-Bu, s-butyl, CH(CH₃)CH₂CH₃), 2-methyl-2-propyl (t-Bu, t-butyl, —C(CH₃)₃), 1-pentyl (n-pentyl, CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃).

In some embodiments, an alkyl may be an “alkylamino” which refers to an amino group substituted with at least one alkyl group. Nonlimiting examples of amino groups include —NH₂, —NH(CH₃), —N(CH₃)₂, —NH(CH₂CH₃), —N(CH₂CH₃)₂, —NH(phenyl), —N(phenyl)₂, NH(benzyl), —N(benzyl)₂, etc. Substituted alkylamino refers generally to alkylamino groups, as defined above, in which at least one substituted alkyl, as defined herein, is attached to the amino nitrogen atom. Non-limiting examples of substituted alkylamino includes —NH(alkylene-C(O)—OH), —NH(alkylene-C(O)—O-alkyl), —N(alkylene-C(O)—OH)₂, —N(alkylene-C(O)—O-alkyl)₂, etc.

In some embodiments, an alkyl may be a “heteroalkyl” which refers to an alkyl group where one or more carbon atoms have been replaced with a heteroatom, such as, O, N, or S. For example, if the carbon atom of the alkyl group which is attached to the parent molecule is replaced with a heteroatom (e.g., O, N, or S) the resulting heteroalkyl groups are, respectively, an alkoxy group (e.g., —OCH₃, etc.), an amine (e.g., —NHCH₃, —N(CH₃)₂, etc.), or a thioalkyl group (e.g., —SCH₃). If a non-terminal carbon atom of the alkyl group which is not attached to the parent molecule is replaced with a heteroatom (e.g., O, N, or S) the resulting heteroalkyl groups are, respectively, an alkyl ether (e.g., —CH₂CH₂—O—CH₃, etc.), an alkyl amine (e.g., —CH₂NHCH₃, —CH₂N(CH₃)₂, etc.), or a thioalkyl ether (e.g., —CH₂—S—CH₃). If a terminal carbon atom of the alkyl group is replaced with a heteroatom (e.g., O, N, or S), the resulting heteroalkyl groups are, respectively, a hydroxyalkyl group (e.g., —CH₂CH₂—OH), an aminoalkyl group (e.g., CH₂NH₂), or an alkyl thiol group (e.g., —CH₂CH₂—SH). A heteroalkyl group can have, for example, 1 to 20 carbon atoms, 1 to 10 carbon atoms, or 1 to 6 carbon atoms. A C₁-C₆ heteroalkyl group means a heteroalkyl group having 1 to 6 carbon atoms.

In some embodiments, an alkyl may be substituted for example, “substituted alkyl”, in which one or more hydrogen atoms are each independently replaced with a non-hydrogen substituent. Typical substituents include, but are not limited to, —X, —R^{b}, —O⁻, ═O, —OR^{b}, SR^{b}, —S⁻, —NR^{b}₂, —N⁺R^{b}₃, ═NR^{b}, —CX₃, —CN, —OCN, —SCN, —N═C═O, —NCS, —NO, —NO₂, ═N₂, —N₃, —NHC(═O)R^{b}, —OC(═O)R^{b}, —NHC(═O)NR^{b}₂, —S(═O)₂—, —S(═O)₂OH, —S(═O)₂R^{b}, —OS(═O)₂OR^{b}, —S(═O)₂NR^{b}₂, —S(═O)R^{b}, —OP(═O)(OR^{b})₂, —P(═O)(OR^{b})₂, —P(═O)(O⁻)₂, —P(═O)(OH)₂, —P(O)(OR^{b})(O⁻), —C(═O)R^{b}, —C(═O)X, —C(S)R^{b}, —C(O)OR^{b}, —C(O)O⁻, —C(S)OR^{b}, —C(O)SR^{b}, —C(S)SR^{b}, —C(O)NR^{b}₂, —C(S)NR^{b}₂, —C(═NR^{b})NR^{b}₂, where each X is independently a halogen: F, Cl, Br, or I; and each R^{b} is independently H, alkyl, aryl, arylalkyl, a heterocycle, or a protecting group or prodrug moiety. Alkylene, alkenylene, and alkynylene groups may also be similarly substituted. Unless otherwise indicated, when the term “substituted” is used in conjunction with groups such as arylalkyl, which have two or more moieties capable of substitution, the substituents can be attached to the aryl moiety, the alkyl moiety, or both.

On some embodiments, an alkyl may be “optionally substituted,” The term “optionally substituted” in reference to a particular moiety of the compounds of the invention (e.g., an optionally substituted alkyl group) refers to a moiety wherein all substituents are hydrogen or wherein one or more of the hydrogens of the moiety may be replaced by substituents such as those listed under the definition of “substituted”.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

The term “halogen” or, alternatively, a “halide” means a fluorine, chlorine, bromine or iodine atom.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: PCR Reaction Using Chemically Modified Blocking Primers to Produce 5′ Sticky Ended DNA Fragments

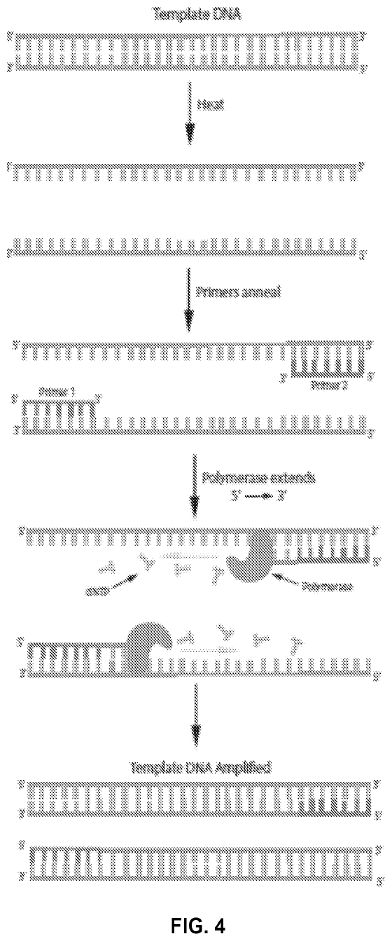

As shown in FIG. 4, using normal primers in a PCR will produce blunt ended DNA fragments. As generally shown in FIG. 5, in one embodiment of the inventive technology, the present inventors generated chemically modified primers, that when used in a standard PCR protocol would result in steric hinderance of the polymerase on the template strand, leading to 5′ single stranded overhangs, thus, the ability to create sticky ended DNA fragments from a PCR.

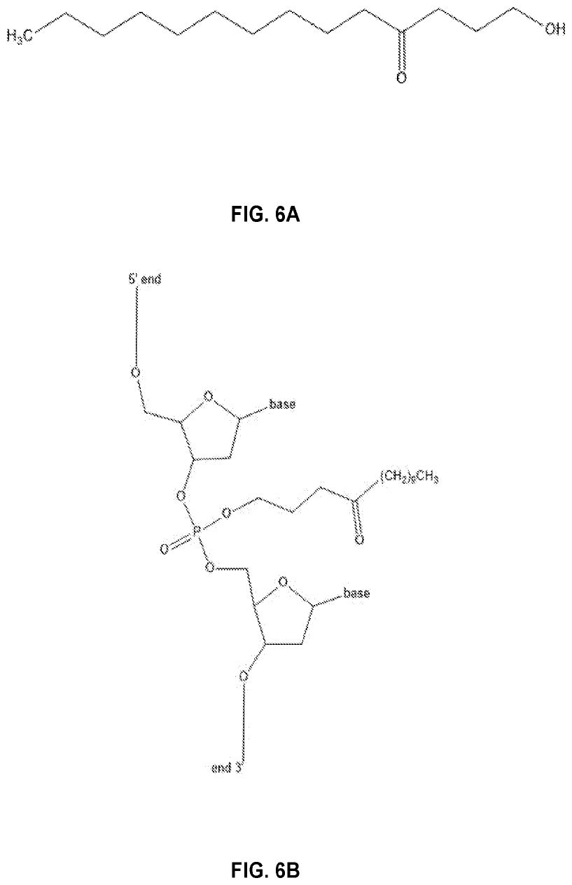

As demonstrated in FIGS. 6A-B, exemplary chemically modified primers, also referred to as blocking primers, were constructed to include a single-oxotetradec-1-yl (OXP) phosphate group modification adhered to the phosphate group on any desired deoxynucleotide of the primer.

### Example 2: Mechanism for OXP Reactivity in Modified Blocking Primer

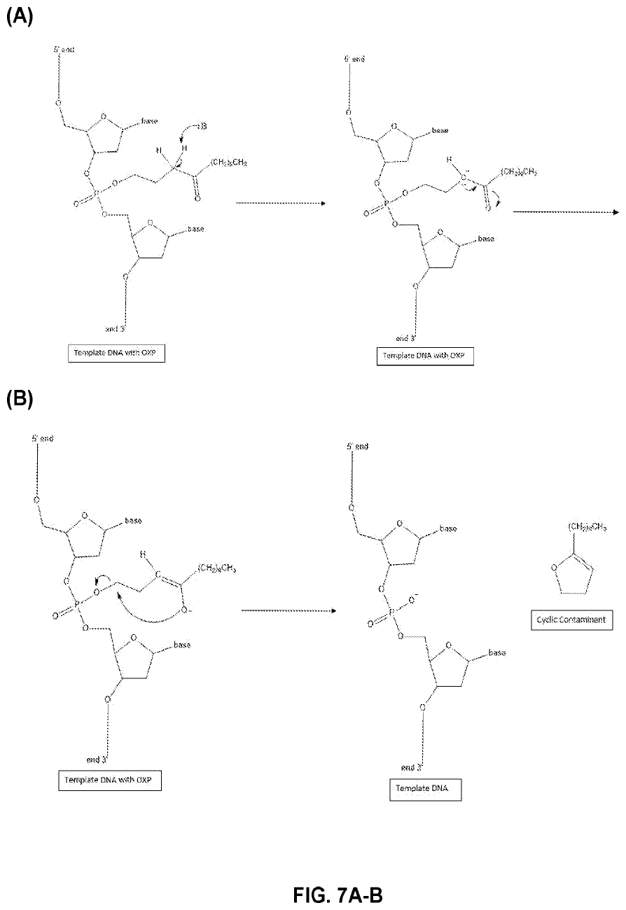

It has been generally indicated that OXP modified primers in a PCR buffer solution at 95° C. have a half-life of around 8.5 minutes. Notably, as outlined in FIG. 7, the OXP group may become reactive at high temperatures resulting in complete dissociation from the DNA phosphate backbone. The buffers used in PCR have an acid-base composition. When heated, the base in the solution may be reacting with the OXP group causing the formation of a carbanion intermediate. The carbanion is unstable, transferring electrons to form a double bond, therefore pushing electrons to the electronegative oxygen. The oxygen, now negative and reactive, may force the OXP modification to rapidly form a favorable 5 ring cyclic compound, cleaving completely from the DNA.

### Example 3: Efficient Polymerase Chain Reaction (PCR) Protocol

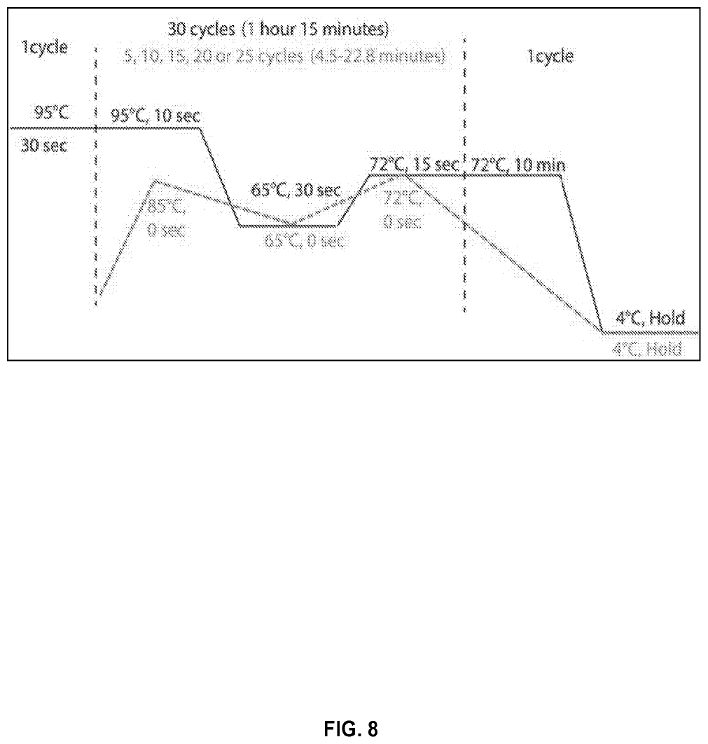

The present inventors designed a PCR using a short DNA fragment, an OXP modified forward primer, and an unmodified reverse primer. Since the OXP modification is thermally labile, the PCR was optimized with low temperatures, decreased times, and limited numbers of cycles. The conditions were effective at amplification while also retaining the stability of the OXP group. Naturally, non-, or less labile chemical groups would not require such modified PCR protocols. As demonstrated in FIG. 8, it was found with the present inventor's design that the thermocycler conditions were optimal at 85° C. for 0 seconds, 65° C. for 0 seconds, 72° C. for 0 seconds; each ran at various numbers of cycles (25, 20, 15, 10 and 5 cycles). The present thermocycler conditions are roughly 16.5 times-3.5 times faster than standard conditions, depending on the number of cycles run.

### Example 4: Mass Spectrometry Analysis of PCR Products

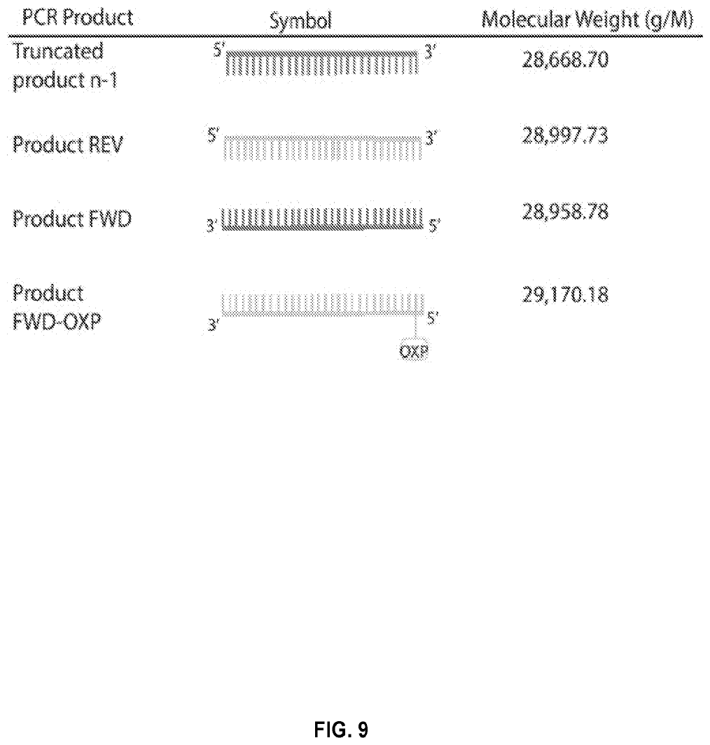

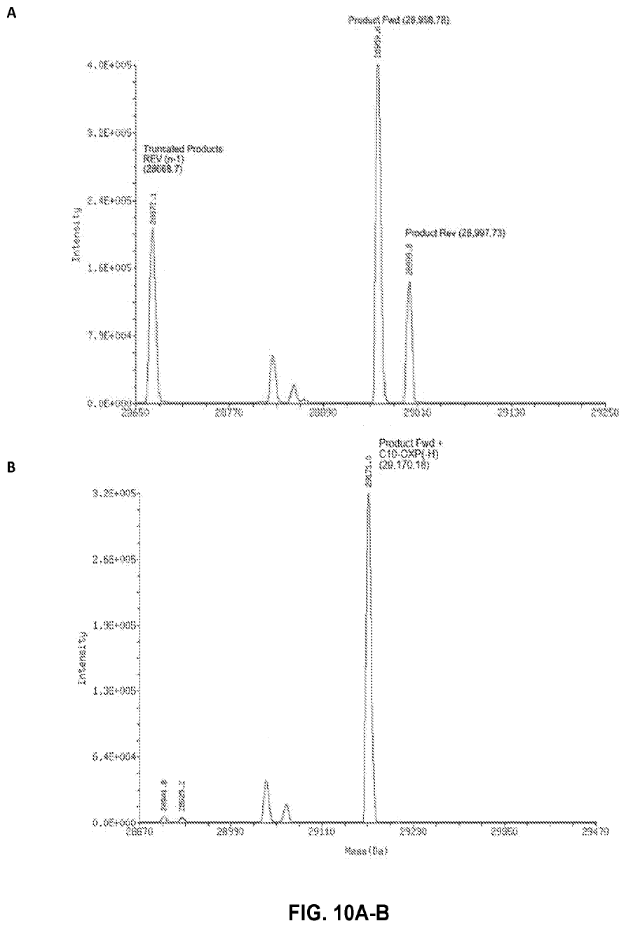

As shown in FIG. 9, mass spectrometry analysis was performed on the PCR products for 5, 10, 15, 20 and 25 cycles to determine if there were overhanging products and where truncation occurred. The data indicated a major product of n−1 truncation of the elongating strand relative to the position of the OXP modification on the opposing DNA strand. Cycle 20 mass spectrometry data is shown in FIG. 10, showing the polymerase had the ability to elongate the DNA strand until the steric hinderance of the OXP on the template strand dislodged the polymerase, preventing any further nucleotide addition to the extending strand. As also shown in FIG. 10, other major PCR products included a forward product with an intact OXP, as this DNA strand is complementary to the n−1 truncated product. Full length reverse product and full length forward product without intact OXP were also noted as a high intensity product. The full length forward and reverse products were expected due to the instability of the OXP primer at high temperatures. When the OXP phosphate modification fell off the DNA, it permitted the polymerase to fully extend the forward strand, which is complementary to the full length reverse strand. The mass spectrometry data indicated that the use of an OXP modified primer in PCR is effective in creating an n−1 truncated product on the elongating strand, relative to the OXP phosphate modification group.

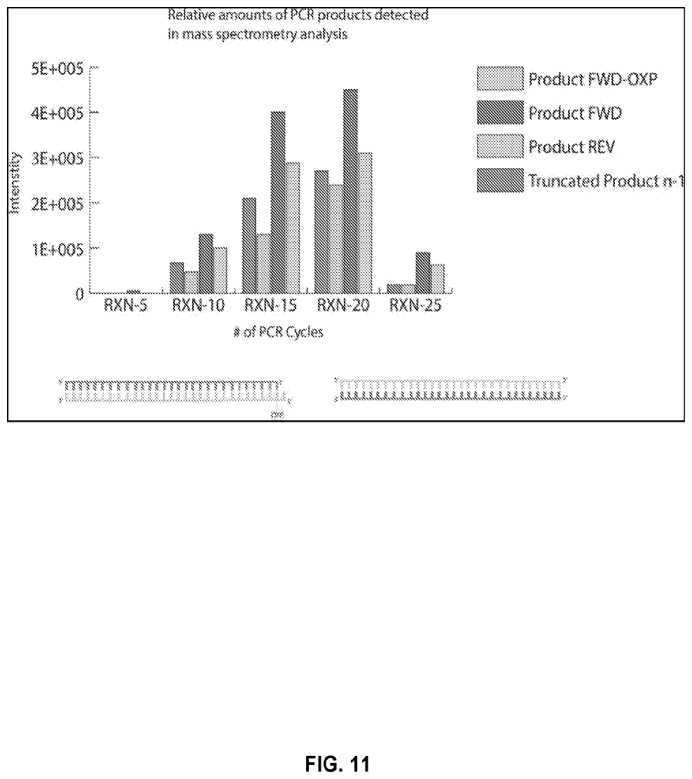

As demonstrated in FIG. 11, the mass spectrometry data from the different cycling conditions had differing results. PCRs that underwent 10, 15, and 20 cycles had similar mass spectrometry data. As cycles increased, there was higher intensity of the n−1 truncated product. The data for the PCR with 5 cycles was inconclusive with various truncated and low intensity products due to an insignificant amount of cycles. The PCR with 25 cycles produced inconsistent results with n−1 truncated product present in very low intensity, along with other varying truncations, most likely due to OXP instability. The optimized conditions, for clear amounts of n−1 truncated product while using an OXP modification, was with a 10-20 cycle PCR.

### Example 5: Polymerase Destabilization with OXP Modifications Present on Chemically Modified Blocking Primer

The 5′ single stranded overhang of the PCR product is due to the polymerase failing to extend the complementary DNA to its full length. In one embodiment, this may be due to the OXP modification sterically hindering the polymerase from progressing along the DNA strand, or it could be due to the exonuclease editing function of the enzyme in alternative embodiments. The present inventors used Phusion polymerase in this embodiment on the invention, which is a family B DNA polymerase. This class of polymerase has very identifiable features, described as a hand with palm, thumb, and finger structures, as well as a 3′ to 5′ exonuclease editing factor. As the complementary DNA strand is being synthesized by the polymerase, the palm holds the template DNA, the thumb locks the DNA in place, and the fingers assist in dNTP incorporation. If an incorrect nucleotide is added, the elongating strand will swing into the exonuclease active site and edits will be made.

Additionally, DNA damage can influence the extension ability of the polymerase, thereby affecting the kinetic balance between polymerase and exonuclease activity. The result could be inhibition of DNA extension, which is most likely the case with a highly effective exonuclease polymerase, like Phusion. The OXP modification on the template DNA strand is foreign and may lead to thermodynamic instability in the polymerase domain. The polymerase active site may lose affinity for the DNA while the exonuclease active site gains affinity, attempting to fix the error. However, the exonuclease does not have the machinery to fix damage on the DNA backbone. The two active sites are idle with the polymerase unable to extend and the exonuclease unable to make edits. Both sites have decreased affinity and the structures dissociate, thereby leading to the inability to extend the elongating strand.

### Example 6: Strategy for Direct Synthesis of Sticky Ended DNA

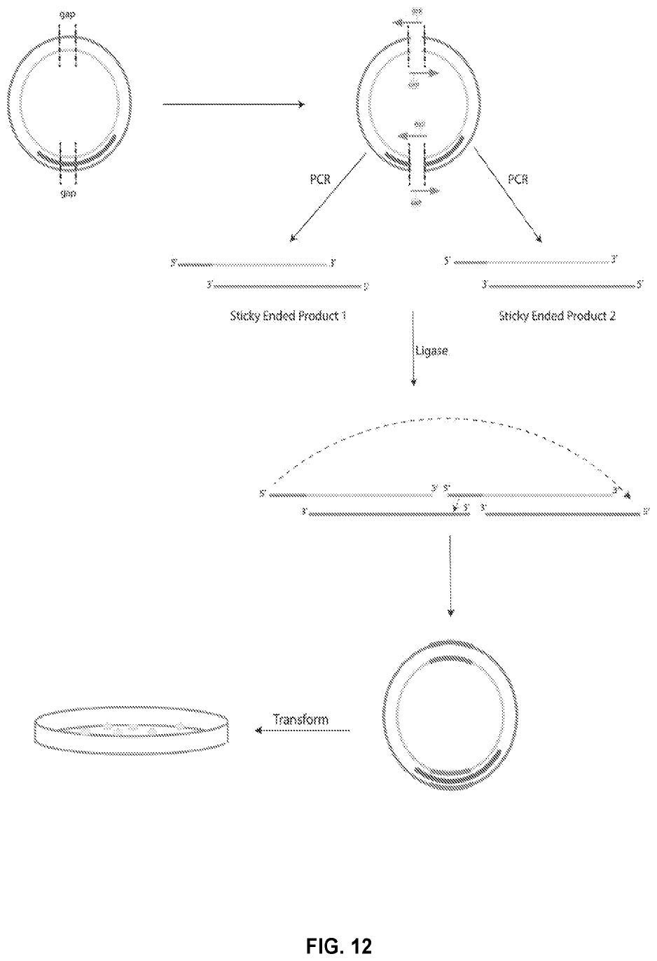

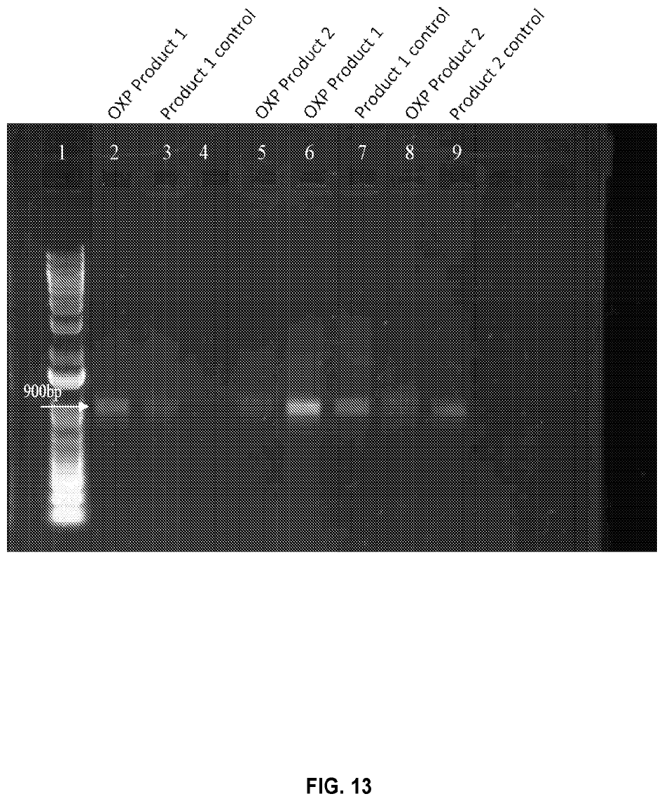

After the present inventor determined the truncation as n−1 on the complementary strand, relative to the OXP modification, the present inventors demonstrated a system to create sticky ended DNA, using OXP modified primers and PCR methods, that could be ligated together to form a functional DNA construct. As shown in FIG. 12, a plasmid was sectioned into two DNA fragments, removing bases needed for chloramphenicol resistance. OXP modified primers were designed flanking the ends of each DNA fragment with overhanging regions that would re-introduce the removed nucleotides. The OXP modification was placed on the phosphate at the 7th nucleotide position of the primer, resulting in 6 base pair overhangs. The PCR was optimized with low temperatures, minimal cycles, and times 8 fold lower than traditional PCRs, to uphold the OXP group's stability. As confirmed in FIG. 13, the resulting product of the PCR was two amplified DNA fragments with 6 base pair, single stranded, overhanging regions at the 5′ ends. The sticky ended DNA products were then ligated together, re-forming the original plasmid. The chloramphenicol resistance was restored, allowing for detection of sticky ended success on proper antibiotic selection plates.

### Example 7: Strategy for Efficient Ligation of Sticky Ended DNA

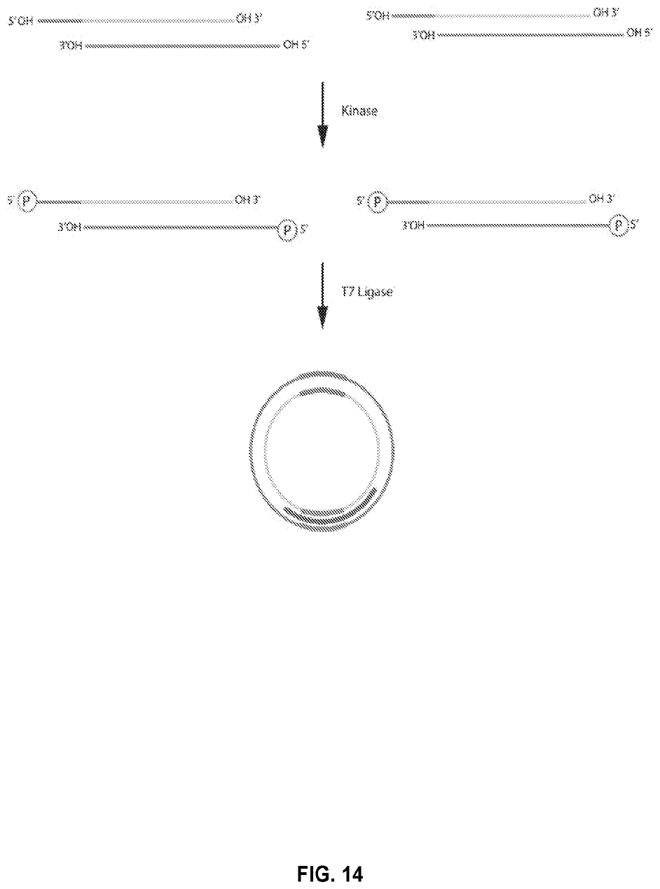

Ligase enzymes utilized the 3′ hydroxyl and 5′ phosphate to form a phosphodiester bond between the two DNA strands, linking the strands together. Notably, here the PCR products had hydroxyl groups on both the 3′ and 5′ ends of the DNA. As further shown in FIG. 14, in one embodiment, a 5′ phosphate was introduced using a kinase enzyme. In the future, the OXP modified primers can be designed with the appropriate 5′ phosphate in place. While the preferred PCR protocol was designed to produce mainly sticky ended products, a certain portion of blunt ended products may be present because of the OXP instability. To ensure only sticky ends were ligated together, the present inventors used a T7 ligase enzyme, which only has the ability to ligate sticky ended DNA. In this manner, the present inventors eliminated any possibility of blunt ended products ligating together.

As outlined in Table 1, the PCRs with 6 and 10 cycles were transformed and plated on chloramphenicol antibiotic plates. Colony growth was noted for DNA produced using OXP modified primers and T7 ligase for both 6 and 10 cycles. Notably, a quantifiable number of colony growth on the OXP produced DNA, with no addition of T7 ligase, for the 6 cycle PCR. This indicated that cloning with the use of ligase was more efficient, however, it was not necessary.

As a result, in one embodiment of the current invention, successful ligation and colony growth without the use of ligase and utilization of the natural repair machinery present in the exemplary bacteria E. coli. In one specific preferred embodiment, variable length 5′ overhangs, such as for example overhangs of 10-15, may be robust in DNA assembly without additions of ligase and using endogenous bacterial repair machinery.

### Example 8: Materials and Methods

PCR of Short DNA Template: The 80 base pair template used was PCRed from a pET32(a) plasmid. The products of the PCR were then gel extracted using a gel extraction kit (Qiagen). The 80 base pair underwent the PCR using the following primers (FWD: TCGCCGCATACACTATTCTC (SEQ ID NO. 1) and REV: CTGTCATGCCATCCGTAAGA (SEQ ID NO. 2). The PCR was done using Phusion High-fidelity PCR Master Mix (ThermoFisher). The product was then gel extracted using a gel extraction kit (Qiagen). The 80 base pair template, reverse primer REV: CTGTCATGCCATCCGTAAGA (SEQ ID NO. 3), and modified OXP primer C*AGAGCAACTCGGTCGCCGCATACACTATTCTC (SEQ ID NO. 4) where the *indicates dA-4-oxotetradec-1-yl (OXP) phosphate group modification were run at various cycles, 5, 10, 15, 20, 25 under the following thermocycler conditions: 85° C. for 0 seconds, 65° C. for 0 seconds, 72° C. for 0 seconds. The products were purified with a phenol chloroform extraction.

Mass Spectrometry: Mass spectrometry was performed by TriLink Biotechnologies.

Crystallography Structures: The crystal structures of polymerases were provided by Kropp et al. and downloaded from protein data bank using accession code “50MF”. Protein structures were assessed using Pymol software.

PCR of Small Plasmid: A 1,869 base pair “small plasmid” was used. 4 standard primers were used to split the small plasmid into two linear fragments and were gel extracted using a gel extraction kit (Qiagen). The OXP modified primers were commissioned by TriLink Biotechnologies:

(SEQ ID NO. 5)
GTTCTT*ACGATGCCATTGGGATATATC
(SEQ ID NO. 6)
ATCAGG***GATAACGCAGGAAAGAACATG
(SEQ ID NO. 7)
AAGAAC**TTTTGAGGCATTTCAGTCAG
(SEQ ID NO. 8)
CCTGAT*CTGTGGATAACCGTAGTCGG

(*) indicates dT4-oxotetradec-1-yl (OXP) phosphate group modification—(**) indicates dA4-oxotetradec-1-yl (OXP) phosphate group modification (***) indicates dG-4-oxotetradec-1-yl (OXP) phosphate group modification. The OXP primers (first and second) were used on one DNA fragment and OXP primers (third and fourth) were used on the other DNA fragment. PCR was done using Phusion High Fidelity Master Mix (Thermo Scientific). PCR conditions with OXP primers were as follows: 95° C. for 0 seconds, 55° C. for 0 seconds, 72° C. for 5 seconds. Cycles varied from 1-10 cycles. PCR products were purified using PCR purification Kits (Qiagen). Purified DNA was then heated at 95° C. for 45 minutes to remove excess OXP chemistries.

Phosphorylation: DNA was phosphorylated using T4 Polynucleotide kinase enzymes and following given protocols (New England BioLabs (NEB)). Ligation: DNA was ligated using T4 DNA ligase or T7 DNA ligase and following given protocols (NEB). Strains: All transformations were completed using 10 ul of chemically competent DH4alpha cells transformed with 0.5 ul of DNA and following given protocols (NEB).

### TABLES

TABLE 1
Comparison of Cloning Efficiencies: With OXP Modifications on the
Oligonucleotide Primers or Without (Standard)

| | Ligase in | PCR | Colony |
|---|---|---|---|
| Primers used in PCR | Transformation | Cycles | Count |
| OXP | + | 6 | 110 |
| OXP | − | 6 | 2 |
| Standard | + | 6 | 0 |
| Standard | − | 6 | 0 |
| OXP | + | 10 | 50 |
| OXP | − | 10 | 0 |
| Standard | + | 10 | 0 |
| Standard | − | 10 | 0 |

### REFERENCES

[1] Agar, N., Halliday, G., Barnetson, R. S. C., and Jones, A. (2003). A novel technique for the examination of skin biopsies by laser capture microdissection. Journal of Cutaneous Pathology 30, 265-270.
[2] Cohen, S. N., Chang, A. C., Boyer, H. W., and Helling, R. B. (1973). Construction of biologically functional bacterial plasmids in vitro. Proceedings of the National Academy of Sciences 70, 3240-3244.
[3] Doherty, A. J., Ashford, S. R., Subramanya, H. S., and Wigley, D. B. (1996). Bacteriophage T7 DNA ligase overexpression, purification, crystallization, and characterization. Journal of Biological Chemistry 271, 11083-11089.
[4] Engler, C., Kandzia, R., and Marillonnet, S. (2008). A one pot, one step, precision cloning method with high throughput capability. PLoS ONE 3, e3647.
[5] Gibson, D. G., Young, L., Chuang, R.-Y., Venter, J. C., Hutchison III, C. A., and Smith, H. O. (2009). Enzymatic assembly of DNA molecules up to several hundred kilobases. Nature Methods 6, 343.
[6] Kennedy, E. M., Hergott, C., Dewhurst, S., and Kim, B. (2009). The mechanistic architecture of thermostable Pyrococcus furiosus family B DNA polymerase motif A and its interaction with the dNTP substrate. Biochemistry 48, 11161-11168.
[7] Khare, V., and Eckert, K. A. (2002). The proofreading 3′4 5′ exonuclease activity of DNA polymerases: a kinetic barrier to translesion DNA synthesis. Mutation Research/Fundamental and Molecular Mechanisms of Mutagenesis 510, 45-54.
[8] Koppolu, V., Vasigala, V. K. (2016). Role of Escherichia coli in Biofuel Production. Microbiology Insights 9, 29-35.
[9] Kropp, H. M., Betz, K., Wirth, J., Diederichs, K., and Marx, A. (2017). Crystal structures of ternary complexes of archaeal B-family DNA polymerases. PloS One 12, e0188005.
[10] Lebedev, A. V., Paul, N., Yee, J., Timoshchuk, V. A., Shum, J., Miyagi, K., Kellum, J., Hogrefe, R. I., and Zon, G. (2008). Hot start PCR with heat-activatable primers: a novel approach for improved PCR performance. Nucleic Acids Res. 36, e131.
[11] Monroe, T., and Haselton, F. (1999). Activating transcription with light using caged DNA. In Proceedings of the First Joint BMES/EMBS Conference. 1999 IEEE Engineering in Medicine and Biology 21st Annual Conference and the 1999 Annual Fall Meeting of the Biomedical Engineering Society (Cat. N, p. 97-vol.)
[12] Mullis, K. B. (1990). The unusual origin of the polymerase chain reaction. Scientific American 262, 56-65.
[13] Poongavanam, V., Madala, P. K., Højland, T., and Veedu, R. N. (2014). Computational investigation of locked nucleic acid (LNA) nucleotides in the active sites of DNA polymerases by molecular docking simulations. PloS One 9, e102126.
[14] Smith, H. O., and Nathans, D. (1973). A suggested nomenclature for bacterial host modification and restriction systems and their enzymes. Journal of Molecular Biology 81, 419-423
[15] Stevenson, J., Krycer, J. R, Phan, L., Brown, A. j. (2013). A Practical Comparison of Ligation-Independent Cloning Techniques. PloS One 8, e83888.
[16] Wang, R-Y., Shin Z-Y., Guo Y-Y., Chen J-C., Chen G-Q. (2013). DNA Fragments Assembly Based on Nicking Enzyme System. PloS One 8, e57943.
[17] Wilk, A., Chmielewski, M. K., Grajkowski, A., Phillips, L. R., and Beaucage, S. L. (2001). The 4-oxopentyl group as a labile phosphate/thiophosphate protecting group for synthetic oligodeoxyribonucleotides. Tetrahedron Letters 42, 5635-5639.
[18] Kuzuya, Akinori et al. “Enzyme treatment-free and ligation-independent cloning using caged primers in polymerase chain reactions.” Molecules (Basel, Switzerland) vol. 17,1 328-40. 30 Dec. 2011, doi:10.3390/molecules170103

SEQUENCE LISTING

Forward primer
Artificial
SEQ ID NO. 1
TCGCCGCATACACTATTCTC
Reverse primer
Artificial
SEQ ID NO. 2
CTGTCATGCCATCCGTAAGA
Reverse primer
Artificial
SEQ ID NO. 3
CTGTCATGCCATCCGTAAGA
Modified primer
Artificial
SEQ ID NO. 4
C*AGAGCAACTCGGTCGCCGCATACACTATTCTC
Modified primer
Artificial
SEQ ID NO. 5
GTTCTT*ACGATGCCATTGGGATATATC
Modified primer
Artificial
SEQ ID NO. 6
ATCAGG***GATAACGCAGGAAAGAACATG
Modified primer
Artificial
SEQ ID NO. 7
AAGAAC**TTTTGAGGCATTTCAGTCAG
Modified primer
Artificial
SEQ ID NO. 8
CCTGAT*CTGTGGATAACCGTAGTCGG
*indicates dT4-oxotetradec-1-yl (OXP) phosphate group modification
**indicates dA4-oxotetradec-1-yl (OXP) phosphate group modification
***indicates dG-4-oxotetradec-1-yl (OXP) phosphate group modification

1-20. (canceled)

21. A method of direct synthesis of sticky ended DNA comprising the steps of:
generating a primer having a nucleotide sequence that anneals to a target region in a template DNA;
generating chemically modified blocking primer having a nucleotide sequence that anneals to a target region in a template DNA and an overhanging region at the 5′ end, and wherein said chemically modified blocking primer incorporates a blocking group acceptor at a chosen position on said nucleotide sequence, wherein said blocking group acceptor is configured to be coupled with a reversible blocking group modification;
coupling a reversible blocking group modification with said blocking group acceptor such that said reversible blocking group modification prevents DNA polymerase from fully extending the complementary strand during PCR amplification resulting in a 5′ overhang;
running a polymerase chain reaction (PCR) protocol with said primer, said chemically modified blocking primer, and a template DNA wherein said end-product of said PCR is a plurality of sticky ended DNA products;
decoupling said blocking group from said plurality of sticky ended DNA products; and
ligating one or more of said plurality sticky ended DNA products together.

22. The method of claim 21, wherein the step of incorporating a blocking group acceptor at a chosen position on said chemically modified blocking primer comprises the step of incorporating a thiophosphate group at a chosen position on said chemically modified blocking primer.

23. The method of claim 22, wherein the step of coupling a reversible blocking group modification with said blocking group acceptor comprises the step of coupling an alkyl halide with said thiophosphate group such that said alkyl halide prevents DNA polymerase from fully extending the complementary strand during PCR amplification resulting in a 5′ overhang.

24. The method of claim 23, wherein said step of coupling an alkyl halide with said thiophosphate group comprises the step of coupling an alkyl halide with said thiophosphate through a SN2 reaction.

25. The method of claim 23, wherein said step coupling is performed after the chemically modified blocking primer is synthesized.

26. The method of claim 24, wherein said alkyl halide comprises a trimethyl lock.

27. The method of claim 26, wherein said trimethyl lock comprises a trimethyl lock having a formula: 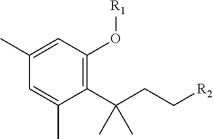
wherein,
R₁ is an R-trigger group selected from the group consisting of: 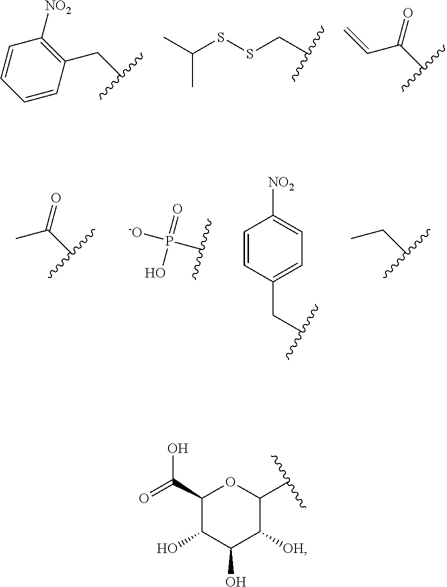
and R₂ is a halide.

28. The method of claim 21, wherein said step of coupling said reversible blocking group modification with said blocking group acceptor comprises the step of coupling a thioesterase or an esterase triggered reversible blocking group modification with said blocking group acceptor.

29. The method of claim 21, wherein said step of coupling said reversible blocking group modification with said blocking group acceptor comprises the step of coupling a β-glucuronidase triggered reversible blocking group modification with said blocking group acceptor.

30. The method of claim 21, wherein said step of ligating said sticky ended DNA products together comprises the step of enzymatically ligating said sticky ended DNA products together.

31. The method of claim 30, wherein said step of enzymatically ligating said sticky ended DNA products together comprises the step of enzymatically ligating said sticky ended DNA products together using a ligase enzyme.

32. The method of claim 21, wherein said step of ligating said sticky ended DNA products together comprises the step of complementarily pairing said overhanging 5′ regions together in vivo.

33-34. (canceled)

35. The method of claim 21, wherein said overhanging region at the 5′ end may be complementary with another overhanging region at the 5′ end on a DNA insert forming a recombinant double stranded DNA molecule.

36. The method of claim 21, further comprising the step of introducing 5′ phosphate to said plurality of sticky ended DNA products.

37. The method of claim 36, wherein said step of introducing 5′ phosphate to said plurality of sticky ended DNA products comprises the step of applying a kinase enzyme that introduces a 5′ phosphate to said plurality of sticky ended DNA products.

38. The method of claim 21, wherein said step of decoupling said blocking group from said plurality of sticky ended DNA products comprises the step selected from the group consisting of: enzymatic deprotection, thermal deprotection, chemical deprotection, catalytic deprotection, photocage deprotection, or other reversible chemistry.

39-49. (canceled)

50. A method of direct synthesis of sticky ended DNA comprising the steps of:
generating a primer having a nucleotide sequence that anneals to a target region in a template DNA;
generating chemically modified blocking primer having a nucleotide sequence that anneals to a target region in a template DNA and an overhanging region at the 5′ end, and wherein said chemically modified blocking primer incorporates a blocking group acceptor at a chosen position on said nucleotide sequence, wherein said blocking group acceptor is configured to be coupled with a reversible blocking group modification;
coupling a reversible blocking group modification with said blocking group acceptor such that said reversible blocking group modification prevents DNA polymerase from fully extending the complementary strand during PCR amplification resulting in a 5′ overhang;
running a polymerase chain reaction (PCR) protocol with said primer, said chemically modified blocking primer, and a template DNA wherein said end-product of said PCR is a plurality of sticky ended DNA products.

51. The method of claim 50, wherein said polymerase chain reaction (PCR) comprises a modified polymerase chain reaction (mPCR).

52. The method of claim 50, and further comprising the step of transforming a cell with said plurality of sticky ended DNA products, wherein said plurality of sticky ended DNA products are ligated together by said cell's endogenous cellular DNA repair machinery forming a recombinant double stranded DNA molecule.

53-54. (canceled)

55. A method of direct synthesis of sticky ended DNA comprising the steps of:
generating a primer having a nucleotide sequence that anneals to a target region in a template DNA;
generating chemically modified blocking primer having a nucleotide sequence that anneals to a target region in a template DNA and an overhanging region at the 5′ end, and wherein said chemically modified blocking primer incorporates a phosphoramidite compound modified with a reversible blocking group modification wherein said reversible blocking group modification prevents DNA polymerase from fully extending the complementary strand during PCR amplification resulting in a 5′ overhang;
running a polymerase chain reaction (PCR) protocol with said primer, said chemically modified blocking primer, and a template DNA wherein said end-product of said PCR is a plurality of sticky ended DNA products;
decoupling said blocking group from said plurality of sticky ended DNA products; and
ligating one or more of said plurality sticky ended DNA products together.

56-77. (canceled)


SEQUENCE LISTING (8 sequences)

SEQ ID NO: 1 - DNA, 20 bp, Artificial Sequence
  Forward primer
tcgccgcatacactattctc

SEQ ID NO: 2 - DNA, 20 bp, Artificial Sequence
  Reverse primer
ctgtcatgccatccgtaaga

SEQ ID NO: 3 - DNA, 20 bp, Artificial Sequence
  Reverse primer
ctgtcatgccatccgtaaga

SEQ ID NO: 4 - DNA, 33 bp, Artificial Sequence
  Modified primer
cagagcaactcggtcgccgcatacactattctc

SEQ ID NO: 5 - DNA, 27 bp, Artificial Sequence
  Modified primer
gttcttacgatgccattgggatatatc

SEQ ID NO: 6 - DNA, 27 bp, Artificial Sequence
  Modified primer
atcagggataacgcaggaaagaacatg

SEQ ID NO: 7 - DNA, 26 bp, Artificial Sequence
  Modified primer
aagaacttttgaggcatttcagtcag

SEQ ID NO: 8 - DNA, 26 bp, Artificial Sequence
  Modified primer
cctgatctgtggataaccgtagtcgg